(19) United States
(12) Patent Application Publication
(10) Pub. No.: US 2012/0052024 A1
(43) Pub. Date: Mar. 1, 2012

(54) Novel Composition

(76) Inventors: Jane Fletcher, Bristol (GB); Christabel Fowler, Surrey (GB); Stephen Mann, Bristol (GB); Charles Richard Parkinson, Surrey (GB); Dominic Walsh, Bristol (GB)

(21) Appl. No.: 13/319,555

(22) PCT Filed: May 12, 2010
(86) PCT No.: PCT/EP10/56607

§ 371 Date: Nov. 9, 2011

(30) Foreign Application Priority Data
May 15, 2009 (GB) 0908433.6

(51) Int. Cl. A61K 8/72; A61Q 11/00; A61K 8/73

(52) U.S. Cl. 424/49

(57) ABSTRACT

Electrospun polymer fibres comprising an amorphous calcium compound, oral care compositions comprising such fibres and their use in remineralising dental hard tissues and/or blocking dentinal tubules are described. Such compositions are of use in strengthening dental enamel of teeth thereby providing protection from acidic challenges. Such compositions are of use in combating dental erosion and/or tooth wear. Such compositions are of use in combating dental caries. Such compositions are of use in combating dentine hypersensitivity.

### FIELD OF THE INVENTION

The present invention relates to electrospun polymer fibres comprising an amorphous calcium compound, oral care compositions comprising such fibres and their use in remineralising dental hard tissues and/or blocking dentinal tubules. Such compositions are of use in strengthening dental enamel of teeth thereby providing protection from acidic challenges. Such compositions are of use in combating (i.e. helping to prevent, inhibit and/or treat) dental erosion and/or tooth wear. Such compositions are of use in combating dental caries. Such compositions are of use in combating dentine hypersensitivity.

### BACKGROUND OF THE INVENTION

Tooth mineral is composed predominantly of calcium hydroxyapatite, Ca₁₀(PO₄)₆(OH)₂, which may be partially substituted with anions such as carbonate or fluoride, and cations such as zinc or magnesium. Tooth mineral may also contain non-apatitic mineral phases such as octacalcium phosphate and calcium carbonate.

Tooth loss may occur as a result of dental caries, which is a multifactorial disease where bacterial acids such as lactic acid produce sub-surface demineralisation that does not fully remineralise, resulting in progressive tissue loss and eventually cavity formation. The presence of a plaque biofilm is a prerequisite for dental caries, and acidogenic bacteria such as Streptococcus mutans may become pathogenic when levels of easily fermentable carbohydrate, such as sucrose, are elevated for extended periods of time.

Even in the absence of disease, loss of dental hard tissues can occur as a result of acid erosion and/or physical tooth wear; these processes are believed to act synergistically. Exposure of the dental hard tissues to acid causes demineralisation, resulting in surface softening and a decrease in mineral density. Under normal physiological conditions, demineralised tissues self-repair through the remineralising effects of saliva. Saliva is supersaturated with respect to calcium and phosphate, and in healthy individuals saliva secretion serves to wash out the acid challenge, and raises the pH so as to alter the equilibrium in favour of mineral deposition.

Dental erosion (i.e. acid erosion or acid wear) is a surface phenomenon that involves demineralisation, and ultimately complete dissolution of the tooth surface by acids that are not of bacterial origin. Most commonly the acid will be of dietary origin, such as citric acid from fruit or carbonated drinks, phosphoric acid from cola drinks and acetic acid such as from vinaigrette. Dental erosion may also be caused by repeated contact with hydrochloric acid (HCl) produced in the stomach, which may enter the oral cavity through an involuntary response such as gastroesophageal reflux, or through an induced response as may be encountered in sufferers of bulimia.

Tooth wear (ie physical tooth wear) is caused by attrition and/or abrasion. Attrition occurs when tooth surfaces rub against each other, a form of two-body wear. An often dramatic example is that observed in subjects with bruxism, a grinding habit where the applied forces are high, and is characterised by accelerated wear, particularly on the occlusal surfaces. Abrasion typically occurs as a result of three-body wear and the most common example is that associated with brushing with a toothpaste. In the case of fully mineralised enamel, levels of wear caused by commercially available toothpastes are minimal and of little or no clinical consequence. However, if enamel has been demineralised and softened by exposure to an erosive challenge, the enamel becomes more susceptible to tooth wear. Dentine is much softer than enamel and consequently is more susceptible to wear. Subjects with exposed dentine should avoid the use of highly abrasive toothpastes, such as those based on alumina. Again, softening of dentine by an erosive challenge will increase susceptibility of the tissue to wear.

Dentine is a vital tissue that in vivo is normally covered by enamel or cementum depending on the location i.e. crown versus root respectively. Dentine has a much higher organic content than enamel and its structure is characterised by the presence of fluid-filled tubules that run from the surface of the dentine-enamel or dentine-cementum junction to the odontoblast/pulp interface. It is widely accepted that the origins of dentine hypersensitivity relate to changes in fluid flow in exposed tubules, (the hydrodynamic theory), that result in stimulation of mechanoreceptors thought to be located close to the odontoblast/pulp interface. Not all exposed dentine is sensitive since it is generally covered with a smear layer; an occlusive mixture comprised predominantly of mineral and proteins derived from dentine itself, but also containing organic components from saliva.

Over time, the lumen of the tubule may become progressively occluded with mineralised tissue. The formation of reparative dentine in response to trauma or chemical irritation of the pulp is also well documented. Nonetheless, an erosive challenge can remove the smear layer and tubule “plugs” making the dentine much more susceptible to external stimuli such as hot, cold and pressure. As previously indicated, an erosive challenge can also make the dentine surface much more susceptible to wear. Progressive dentine wear can result in an increase in hypersensitivity, especially in cases where dentine wear is rapid.

Loss of the protective enamel layer through erosion and/or acid-mediated wear will expose the underlying dentine, and are therefore primary aetiological factors in the development of dentine hypersensitivity.

There are two categories of therapy for the treatment of dentine hypersensitivity based upon two modes of action. The first category, nerve-depolarising agents, are pharmaceutical agents such as potassium nitrate, which function by interfering with neural transduction of the pain stimulus.

The second category, known as occluding agents, function by physically blocking the exposed ends of the dentinal tubules, thereby reducing dentinal fluid movement and reducing the irritation associated with the shear stress described by the hydrodynamic theory.

The occlusion approach typically involves treating the tooth with a chemical or physical agent that creates a deposition layer within or over the dentine tubules. This layer mechanically occludes the tubules and prevents or limits fluid movement within the tubule to such an extent that stimulation of the neuron is not achieved. Examples of occlusion actives include among others, calcium salts, oxalate salts, stannous salts, glasses, and varnishes.

U.S. Pat. No. 5,037,639, U.S. Pat. No. 5,268,167, U.S. Pat. No. 5,437,857, U.S. Pat. No. 5,460,803 and U.S. Pat. No. 5,534,244 (all assigned to ADAHF) describe various amorphous calcium compounds for use in remineralising teeth. It is suggested in these patents that such amorphous calcium compounds or solutions which form these amorphous calcium compounds can help prevent or repair dental weaknesses such as dental caries, exposed roots and dentine hypersensitivity. It is claimed that such compounds have high solubilities and, in the aqueous environment of the mouth, have fast conversion rates to less soluble apatite structures, which can help remineralise teeth.

The present invention is based on the discovery that such amorphous calcium compounds can be stabilised against premature conversion to apatite structures if they are incorporated within an electrospun polymer fibre. Such electrospun fibres can be formulated in oral compositions providing a stable form of an amorphous calcium compound, ready to be destabilised at the tooth surface, and are of use in remineralising dental hard tissues and/or for blocking dentinal tubules.

### SUMMARY OF THE INVENTION

Accordingly, in a first aspect, the present invention provides an electrospun polymer fibre comprising an amorphous calcium compound.

In a second aspect the present invention provides an oral care composition comprising an electrospun polymer fibre comprising an amorphous calcium compound and its use in remineralising dental hard tissues and/or blocking dentinal tubules.

Such compositions are of use in strengthening dental enamel of teeth thereby providing protection from acidic challenges. Such compositions are of use in combating (i.e. helping to prevent, inhibit and/or treat) dental erosion and/or tooth wear. Such compositions are of use in combating dental caries. Such compositions are of use in combating dentine hypersensitivity.

In another aspect the present invention provides a method of remineralising dental hard tissues and/or blocking dentinal tubules in a patient in need thereof which comprises administering an effective amount of an oral care composition comprising an electrospun polymer fibre comprising an amorphous calcium compound. In another aspect the present invention provides the use of an electrospun polymer fibre comprising an amorphous calcium compound for the manufacture of an oral care composition for use in remineralising dental hard tissues and/or blocking dentinal tubules.

### DETAILED DESCRIPTION OF THE INVENTION

Suitable amorphous calcium compounds are known from the above noted ADAHF patents. Examples of suitable amorphous calcium compounds include amorphous calcium phosphate (ACP), amorphous calcium phosphate fluoride (ACPF), amorphous calcium carbonate phosphate (ACCP), amorphous calcium carbonate phosphate fluoride (ACCPF), amorphous calcium fluoride and strontium doped derivatives thereof including amorphous strontium calcium phosphate (ASCP), amorphous strontium calcium phosphate fluoride (ASCPF), amorphous strontium calcium carbonate phosphate (ASCCP), amorphous strontium calcium carbonate phosphate fluoride (ASCCPF) or a mixture thereof.

Suitably the amorphous calcium compound is ACP or ASCP or a mixture thereof.

The amorphous calcium compound can be prepared using known methods for example as described in the above noted ADAHF patents, or as described in Li et al. Materials Science and Technology, 20, 2004, 1075 to 1078 or in Li et al, J. Materials Science Letters, 22, 2003, 1015-1016 which latter document describes the preparation of an amorphous calcium compound (ACP) stabilised during its preparation with polyethylene glycol or polyvinyl alcohol.

Electrospun polymer fibres are known and can be prepared by electrospinning polymers or polymer solutions, for example as described in a review paper by Greiner et al, Angew. Chem. Int. Ed. 2007, 46, 5670-5703, and references described therein. Electrospinning is a technique which can be used to spin polymer fibres in the range of nanometres to a few microns.

Suitably the electrospun polymer fibre of the present invention has a diameter in the range from 10 nm to 10 μm, for example from 50 nm to 5 μm, suitably from 100 nm to 1 μm. Suitable polymers for electrospinning are described in the above-noted Greiner et al review paper and include those that are non-toxic and substantive to the tooth surface so that they can adhere to or form a gel on the tooth surface and thereby enhance delivery of an amorphous calcium compound on to or into the tooth surface.

Examples of suitable polymers include a polyvinyl pyrrolidone (PVP) or a derivative thereof, a polysaccharide, a cellulose polymer, an anionic polymer, a biopolymer, a bioerodible polymer, a polyethylene oxide, a polyvinyl alcohol, or an acrylamide copolymer, or a mixture thereof.

Suitably the polymer is PVP or a derivative thereof including a vinylpyrrolidone vinyl acetate copolymer (VP/VA) or a vinylpyrrolidone vinyl alcohol copolymer (VP/VOH) or a mixture thereof

Suitably the polymer is a polysaccharide, examples of which include a dextran, an alginate, a pullulan, or a xyloglucan, or a mixture thereof.

Suitably the polymer is a cellulose polymer, examples of which include a (C₁₋₆)alkylcellulose ether, for instance methylcellulose; a hydroxy(C₁₋₆)alkylcellulose ether, for instance hydroxyethylcellulose or hydroxypropylcellulose; a (C₂₋₆)alkylene oxide modified (C₁₋₆)alkylcellulose ether, for instance hydroxypropylmethylcellulose; or a carboxy(C₁₋₆)alkylcellulose, for instance a carboxymethyl cellulose, or a mixture thereof.

Suitably the polymer is an anionic polymer, by which is meant a polymer comprising a plurality of anionic functional groups which may be the same or different.

In one embodiment the anionic polymer is a polycarboxylate, comprising a plurality of carboxy functional groups, examples of which include a polyacrylic acid, a copolymer of acrylic acid and maleic acid, a copolymer of methacrylic acid and acrylic acid, a copolymer of an alkyl vinyl ether and maleic acid or anhydride, or a copolymer having repeated units of a hydrophilic monomer selected from a carboxylic acid, a dicarboxylic acid or a dicarboxylic acid anhydride and a hydrophobic monomer consisting of an alpha-olefin having at least eight carbon atoms, full and partially hydrolysed forms thereof and full and partial salts thereof. The latter copolymer is described in U.S. Pat. No. 6,241,72 (Block). An example of such a polycarboxylate is PA-18 which is an alternating copolymer of a 1:1 molar ratio of maleic anhydride and 1-octadecene (referred to as octadecene maleic anhydride copolymer).

Suitably a polycarboxylate is a polyacrylic acid, for example having a molecular weight of about 1,000 to about 1,000,000, for example from about 10,000 to about 100,000, or from about 20,000 to 50,000. Suitably the polyacrylic acid may be in a neutralised form, for example in the form of a sodium or potassium salt.

Suitably the polymer is a biopolymer, examples of which include a collagen or a hydrolysate thereof (eg gelatin), an elastin or a hydrolysate thereof, silk, fibrinogen, chitin or chitosan, or a mixture thereof.

Suitably the polymer is a bioerodible polymer, examples of which include a polylactide, a polyglycolic acid, a polycaprolactone, a polyhydroxybutyrate, or a polyester urethane, or a copolymer or block copolymer of such bioerodible polymers, or a mixture thereof.

Suitably the polymer is an acrylamide copolymer, examples of which include poly(acrylamide-co-acrylic acid) or poly(acrylic acid-co-maleic acid)

The electrospun polymer fibres of the present invention may be prepared by electrospinning a solution of a polymer in a suitable solvent such as water or more suitably an organic solvent, for example a C₁₋₆alkanol (such as ethanol) or a halogenated hydrocarbon (such as dichloromethane or chloroform), containing a suspension of an amorphous calcium compound. Other suitable organic solvents include esters such as ethyl acetate, aromatic hydrocarbons such as toluene or xylene, ketones such as acetone or butanone, nitriles such as acetonitrile, or ethers such as tetrahydrofuran or diethyl ether.

Suitably a polymer solution comprising the amorphous calcium compound is placed in a syringe and driven to the end of a metal needle by a syringe pump, where a droplet is formed. When a high voltage is applied the droplet is stretched into a so-called Taylor cone. When the repulsive electrostatic force overcomes surface tension a polymer jet is formed, which is whipped by the electrostatic repulsion and is deposited on to an earthed target in the form of an electrospun mat comprising electrospun polymer fibres comprising the amorphous calcium compound. This mat can be collected and used to prepare an oral care composition of the present invention.

Suitably the amorphous calcium compound is present in an effective amount to remineralise dental hard tissues and/or block dentinal tubules. An effective amount can be determined, for example, by using the methods described in the Examples herein.

Suitably the amorphous calcium compound is present in the electrospun polymer fibre in an amount of at least 10 wt % relative to the polymer, for example at least 20 wt %, at least 30 wt %, at least 40 wt %, at least 50 wt % or at least 60 wt %.

In one embodiment the electrospun mat comprising the electrospun fibres of the present invention can be cut into a strip or a patch and used as a dental strip or dental patch for direct application to the teeth.

Alternatively the electrospun mat can be processed into fragments of electrospun polymer fibres of the present invention, for example by micronisation, which fragments, if desired, can be stored in a C₂₋₆alkanol (eg ethanol) or other non-aqueous solution or gel prior to formulation. These fragments can then be incorporated into an oral care composition further comprising an orally acceptable carrier or excipient.

Therefore, it is to be understood that an electrospun fibre of the present invention may be in the form of an electrospun mat which can be appropriately shaped for oral care use or may be in the form of fragments thereof which can be incorporated into an oral care composition.

Compositions of the present invention are typically formulated in the form of toothpastes, sprays, mouthwashes, gels, lozenges, chewing gums, tablets, pastilles, instant powders, dental strips and dental patches.

Suitable orally acceptable carriers or excipients include abrasives, surfactants, thickening agents, humectants, flavouring agents, sweetening agents, opacifying or colouring agents, preservatives and water, selected from those conventionally used in the oral care composition art for such purposes.

Oral care compositions of the present invention may comprise one or more active agents conventionally used in oral healthcare compositions, for example, a fluoride source, a desensitising agent, an anti-plaque agent, an anti-calculus agent, a whitening agent, an oral malodour agent or a mixture of at least two thereof. Such agents may be included at levels to provide the desired therapeutic effect.

Such active agents may be incorporated directly into the electrospun fibres of the present invention during the preparation of such fibres or they may be added to an oral care composition together with pre-formed electrospun fibres and any carriers or excipients.

Suitable oral care actives and orally acceptable carriers or excipients are described for example in WO 2008/057136 (Procter & Gamble) or EP 929287 (SmithKline Beecham).

Suitably the oral care composition further comprises a source of fluoride ions. Examples of a source of fluoride ions include an alkali metal fluoride such as sodium fluoride, an alkali metal monofluorophosphate such a sodium monofluorophosphate, stannous fluoride, or an amine fluoride in an amount to provide from 25 to 3500 pm of fluoride ions, preferably from 100 to 1500 ppm. A suitable fluoride source is an alkali metal fluoride such as sodium fluoride, for example the composition may contain 0.1 to 0.5% by weight of sodium fluoride, eg 0.205% by weight (equating to 927 ppm of fluoride ions), 0.2542% by weight (equating to 1150 ppm of fluoride ions) or 0.315% by weight (equating to 1426 ppm of fluoride ions).

Compositions of the present invention may further comprise a desensitising agent for combating dentine hypersensitivity. Examples of desensitising agents include a tubule blocking agent or a nerve desensitising agent and mixtures thereof, for example as described in WO 02/15809. Suitable desensitising agents include a strontium salt such as strontium chloride, strontium acetate or strontium nitrate or a potassium salt such as potassium citrate, potassium chloride, potassium bicarbonate, potassium gluconate and especially potassium nitrate.

A desensitising amount of a potassium salt is generally between 2 to 8% by weight of the total composition, for example 5% by weight of potassium nitrate can be used.

In order to minimise any premature conversion of the amorphous calcium compounds to hydroxyapatite structures during storage and prior to use, the compositions of the present invention may be formulated with low amounts of unbound water (suitably less than 20%, for example less than 10% or less than 5% by weight of the total composition) or they may be anhydrous (ie non-aqueous) essentially containing zero amounts of unbound water.

Examples of suitable anhydrous compositions that may comprise the electrospun fibres of the present invention include those described in WO 02/38119 (SmithKline Beecham) and U.S. Pat. No. 5,882,630 (SmithKline Beecham).

Compositions of the present invention may be prepared by admixing the ingredients in the appropriate relative amounts in any order that is convenient and if necessary adjusting the pH to give a desired value for example from 5.5 to 9.0.

The invention is further illustrated by the following Examples.

### EXAMPLE 1

### Electrospun Mat Comprising ACP and PVP

a) Amorphous Calcium Phosphate (ACP)

CaCl₂ (4.44g) and polyethylene glycol, PEG, (17.76 g, molecular weight 8000) were dissolved in distilled water (400 ml) to form a 0.1M solution. A sodium phosphate solution was prepared by adding Na₃PO₄ (4.36 g) to distilled water (200 ml). Both solutions were cooled to approximately 5° C. The Na₃PO₄ solution was added to the CaCl₂/PEG solution. Reaction occurred at approximately 5° C. under stirring for 30 mins. ACP precipitates were obtained by washing the precipitates repeatedly with water to remove the unwanted ions (Na⁺ and Cl⁻) and then ethanol. This method is based upon that described in the above-noted Li et al papers to prepare ACP stabilized with PEG which absorbs to ACP particles decreasing their solubility and inhibiting their transformation to hydroxyapatite.

b) Electrospinning of ACP/PVP Mat

The prepared ACP powder (0.5 g) was added to 2.5 ml ethanol and sonicated for 1 hour. Polyvinyl pyrrolidone, PVP, (0.33 g, molecular weight ˜1,300,000 g/mol) was dissolved in the ACP/ethanol suspension. The polymer solution was placed in a 10 ml syringe fitted with a 19 G (1.1 mm) needle. Electrospinning was carried out at room temperature with a working distance of 8 cm and an applied voltage of 30 kV. The electrospun mat was collected on aluminium foil on the collecting plate, and comprised electrospun PVP fibres containing 60 wt. % of ACP.

### EXAMPLE 2

### Electrospun Mat Comprising ASCP and PVP

a) Amorphous Strontium Calcium Phosphate (ASCP)

Strontium doped ACP (ie ASCP) was prepared following the method described in Example 1a) but by replacing 25 wt % of the CaCl₂ with SrCl₂.6H₂O.

b) Electrospinning of ASCP/PVP Mat

ASCP was electrospun with PVP using the method described in Example 1b), to afford an electrospun mat comprising electrospun PVP fibres containing 60 wt. % of ASCP.

### EXAMPLE 3

### Electrospun Mat Comprising ACP and Dextran

The prepared ACP powder (0.3 g) was added to 3.5 ml water and sonicated for 5 minutes. Dextran (1.88 g, molecular weight ˜500,000 g/mol) was dissolved in the ACP/water suspension. The polymer solution was placed in a 10 ml syringe fitted with a 19 G (1.1 mm) needle. Electrospinning was carried out at room temperature with a working distance of 8 cm and an applied voltage of 25 kV. The electrospun mat was collected on aluminium foil on the collecting plate, and comprised electrospun dextran fibres containing 14 wt. % of ACP.

### EXAMPLE 4

### Treatment (Remineralisation) of Enamel Samples with Electrospun ACP/PVP Mats or with ASCP/PVP Mats

Tooth samples were cut from the sides of sound bovine molars. They were placed in polyurethane moulds measuring 8×5×2 mm and embedded in epoxy resin (Hitek Electronic Materials, Scunthorpe, UK) for 24 hours. Samples were ground and polished using a polishing unit (Kemet International, Maidstone, UK) and silicon carbide disks of up to 1200 grit. This process removed the surface layer of enamel which may have been chemically altered by processes in the oral environment and also exposed a smooth, flat surface for analysis.

Enamel samples were etched for 15 minutes in citric acid (1 wt %). A section of the enamel was then coated in Pt/Pd, leaving the other section free from coating. A droplet of artificial saliva solution containing 300 ppm fluoride was added to the enamel which was then treated with active (ACP/PVP mat or ASCP/PVP mat) and placed in a humid environment for 1 hour. The treated enamel was washed with deionized water for 1 hour, left to dry in air and then coated in Pt/Pd for viewing on a scanning electron microscope (SEM).

(The composition of artificial saliva solution was as follows: Magnesium chloride 0.2 mM, Calcium chloride dihydrate 1 mM, HEPES 20 mM, Potassium dihydrogen orthophosphate 4 mM, Potassium chloride 16 mM, Ammonium chloride 4.5 mM. The pH was adjusted to pH 7 with 1M Potassium hydroxide. Sodium fluoride (300 ppm) was added to the solution.)

An SEM image of the enamel surface after citric acid etching revealed the internal rod structure of the enamel. After treatment of the enamel surface with an ACP/PVP electrospun mat for 1 hour, followed by washing, a new extensive surface coating of granular materials was visible using SEM.

Powder x-ray diffraction (PXRD) of the enamel after etching showed the crystalline nature of the native enamel, hydroxyapatite peaks being clearly visible. An increase in the intensity of the peaks was observed after remineralisation.

A cross-section of the enamel surface was taken using a Focussed Ion Beam (FIB) of gallium ions to etch away a cross section of the sample for viewing under SEM. A section of the same tooth was also treated with the ACP/PVP mats, which was shown to deposit approximately 500 nm of material on the tooth surface during the remineralisation step and following washing.

In a second experiment bovine dental enamel was demineralised with citric acid for 15 minutes, and then treated with the strontium doped-ACP/PVP electrospun mats.

Comparison of before and after treatment with strontium doped ACP/PVP mats indicated a coating of a layer of differing texture to the untreated enamel. Elemental analysis of the enamel surface was carried out using EDXA (Energy Dispersive X-ray Analysis) before and after treatment. The results show the absence of a strontium peak from the untreated enamel and the presence of a strontium peak for the enamel which has been treated with strontium doped-ACP electrospun mat. This suggests that strontium-ACP from the electrospun mat is incorporated into the enamel structure.

Overall the results suggest that placing the ACP/PVP or ASCP/PVP mat upon the enamel surface under humid conditions leads to the release of poorly crystalline calcium phosphate at the enamel surface by dissolution of the mats. Rapid crystallization of the calcium phosphate, promoted by the presence of fluoride occurred at the enamel surface resulting in granular crystal coating that adhered firmly to the enamel.

In conclusion the citric acid etched dental enamel was effectively remineralised after treatment with ACP/PVP or ASCP/PVP electrospun mats and fluoride solution (300 ppm) for one hour. The incorporation of mineral ions from the electrospun mat was confirmed by doping the ACP with Strontium. This marker was clearly visible in the EDXA of the treated enamel sample. The remineralised layer was found to be approximately 500nm thick. This calcium phosphate coating has excellent biocompatibility with enamel.

Electron microscopy. Images of the treated dental enamel were acquired using a field emission scanning electron microscope (FEG-SEM), JEOL JSM 6330F. Prior to imaging, the samples were mounted on aluminium stubs with carbon sticky pads; they were then sputtered with 15 nm thick Pt/Pd for conductivity.

Powder x-ray diffraction (PXRD). X-ray diffractograms of samples were obtained from a D8 Advance Powder X-ray Diffractometer. The diffraction intensity was collected from 10 to 60° at 0.05 degree intervals.

Energy Dispersive X-ray Analysis (EDXA). EDXA was carried out using an Oxford Instruments X-ray Analysis 300 spectrometer.

### EXAMPLE 5

### Treatment (Remineralisation) of Enamel Samples with Electrospun ACP/Dextran Mats

Bovine dental enamel was demineralised with citric acid for 15 minutes, and then treated with the ACP/dextran electrospun mats following the procedure described in Example 4.

Using SEM a slight change in the enamel surface before and after treatment with the ACP/dextran mat was observed, suggesting this mat has had a remineralising effect on the enamel surface.

A rehardening study into the effect of ACP-dextran mats on remineralisation was carried out. The enamel samples were etched with citric acid (1 wt %) for 30 minutes, washed with deionized water and baseline hardness values were measured. The enamel samples were then treated with the test active for 45 minutes and placed in artificial saliva for 40 hours. Hardness values were then taken as summarised in Graph 1. The results from this indicate that the ACP/Dextran mats have a positive impact on the remineralisation of dental enamel.

### EXAMPLE 6

### Treatment of Dentine with Electrospun ACP/PVP Mat

Bovine teeth were demineralised with citric acid for 45 minutes, to reveal the dentine structure which is located below the enamel surface. The dentine was then treated with the ACP/PVP electrospun mats, following the procedure as described in Example 4. Using SEM a change in the dentine surface before and after treatment with the ACP/PVP mat was observed, suggesting this mat has had a remineralising effect on the dentine surface and can effectively fill the dentinal tubules with calcium phosphate.

### EXAMPLE 7

### Further Studies with ACP/PVP or ASCP/PVP Mats

Methods

Preparation of Amorphous Calcium Phosphate (ACP) and Strontium-Doped Amorphous Calcium Phosphate (ASCP)

ACP

The method of preparation of ACP was based on that described by Li et al. (2003) Journal of Materials Science Letters 22 (14) 1015-1016. The preparation was followed apart from a few changes which were made to the drying method.

Calcium chloride (2.22 g) and poly(ethylene glycol) (8.88 g) were dissolved in distilled water (200 ml) to form a 0.1 M solution. A 0.133 M sodium phosphate solution was prepared by adding 2.18 g Na₃PO₄ to distilled water (100 ml). The solutions were cooled to approximately 5° C., the sodium phosphate solution was then added to the calcium chloride/PEG solution and the reaction occurred at approximately 5° C. under vigorous stirring for 30 minutes.

The ACP precipitates were obtained by centrifuging the samples for 3 minutes at 4000 rpm; the supernatant was removed and the precipitates were washed with water to remove the residual ions present (Na+ and Cl−). This procedure was then repeated using ethanol instead of water to wash the samples. Precipitates were left to dry in air, collected and ground into a fine powder using a mortar and pestle.

ASCP

The method of preparation of ASCP (Sr-doped ACP) was followed as described for ACP however, strontium chloride hexahydrate (1.22 g) was added to the PEG solution as well as calcium chloride (3.32 g) prior to mixing with the phosphate solution.

Preparation of Electrospun Mats

PVP/ACP solutions were prepared by adding ACP particles to ethanol followed by thorough mixing and sonication for one hour. The PVP (Mw ˜1,300,000 g.mol-1 used as standard) was then added to the solution and mixed thoroughly. ACP particles were incorporated into the PVP solution in varying ratios as outlined in Table 1.

TABLE 1
ACP/PVP solutions prepared for electrospinning

| ACP wt % relative to PVP | ACP/g | PVP/g | Ethanol/ml |
|---|---|---|---|
| 0 wt % | 0.00 | 0.50 | 5.00 |
| 10 wt % | 0.06 | 0.50 | 5.06 |
| 33 wt % | 0.25 | 0.50 | 5.25 |
| 50 wt % | 0.50 | 0.50 | 5.50 |
| 60 wt % | 0.75 | 0.50 | 5.75 |

Slight increases were made to the amount of ethanol in the solution in an effort to maintain a similar viscosity of solution.

Solutions were placed into the syringe and delivered to the end of the delivery needle (inner diameter 0.3 mm) using a programmable syringe pump. The flow rate of the solution was adjusted to optimize spinning A high voltage supply was used to maintain the voltage in the range 25-30 kV. The nanowires (electrospun polymer fibres) formed were collected on a conductive aluminium plate.

Preparation of Dentine Samples

Dentine samples were cut from the sides of bovine and human molars. Samples were ground and polished using a polishing unit (Kemet International, Maidstone, UK) and silicon carbide disks of up to 1200 grit. This process removed the surface layer of dentine which exposed a smooth, flat surface for analysis. Dentine samples were firstly etched for 10 minutes in a 1 wt % citric acid solution (pH 3.2) and then washed thoroughly in deionized water and left to dry.

Dentine Treatment

A droplet of artificial saliva solution containing 300 ppm fluoride was added to the dentine (50 μl) which was then treated with the electrospun mat (0.01 g) and placed in a humid environment. The treated dentine was washed with deionized water for 1 hour, left to dry in air and then coated in Pt/Pd for viewing on SEM.

Composition of Artificial Saliva Solution:

Magnesium chloride 0.2 mM, calcium chloride dihydrate 1 mM, HEPES 20 mM, potassium dihydrogen orthophosphate 4 mM, potassium chloride 16 mM, ammonium chloride 4.5 mM. The pH was adjusted to pH 7 with 1 M potassium hydroxide. Sodium fluoride (300 ppm) was added to the solution.

Acid Challenge Studies

Following dentine treatment with the electrospun mat and thorough washing, the samples were placed in a 1 wt % citric acid solution (pH 3.2) for 15 minutes. The samples were washed thoroughly and air dried before being coated with Pt/Pd for viewing on the SEM.

Mechanical Challenge Studies

Following dentine treatment with the electrospun mat the samples were washed under a stream of pressurized deionized water for 2 minutes. The samples were air dried before being coated with Pt/Pd for viewing on the SEM.

As a control, the dentine samples were washed in 500 ml of gently stirring deionized water for 60 minutes.

Hydraulic Conductance Studies

Human dentine samples were etched in 10 wt % citric acid solution for 2 minutes, washed thoroughly in deionized water and then placed in the H.C. system. Earle's balanced salt solution was used as the fluid for the system. The fluid flow was measured three times and recorded so an average value of flow rate could be taken. Following this, the dentine sample was treated with an electrospun mat (0.005 g) and hydrated with Earle's balanced salt solution for 20 minutes. The fluid flow was then measured three times and an average value taken. The electrospun mats investigated were a 0 wt % and 60 wt % ACP mat.

Dissolution Rate of Electrospun Mats

The dissolution of PVP powder of varying molecular weights was investigated. PVP powder (0.1 g) was placed in deionized water (10 ml) and stirred at a constant rate. The time it took for the powder to completely dissolve was then recorded.

Electrospun mats of PVP were prepared using varying molecular weights of PVP (Mw 29,000, Mw 40,000 and Mw 1,300,000 g.mol-1). These electrospun mats (0.1 g) were once again placed into deionized water (10 ml) and the time taken to completely dissolve was recorded.

Results and Discussion

The Effect of ACP Content of Electrospun Mats on Calcium Phosphate Deposition and Tubular Occlusion

From SEM images of dentine prior to treatment, the empty tubules that pattern the surface were observed. After treatment with an electrospun ACP (60 wt %) mat for one hour, the dentinal tubules were seen to be effectively occluded with calcium phosphate material using SEM.

In order to establish how much ACP is needed within the electrospun mats to maximise calcium phosphate deposition and tubule occlusion, electrospun mats with varying amounts of ACP were prepared and tested on dentine samples. All samples were hydrated with an artificial saliva solution to hydrate the electrospun mats and all samples had a treatment time of 1 hour.

SEM images were obtained of dentine samples after treatment with electrospun mats containing 0 wt % ACP. As would be expected, no tubular occlusion was observed and there was no evidence of material deposition on the surface; the dentine appeared similar in structure to the dentine substrate prior to treatment. This observation supports the idea that it is the calcium phosphate within the mats which is being deposited within the dentinal tubules and not the PVP polymer.

Dentine substrates treated with electrospun mats containing 10 wt % ACP, exhibited a small amount of material deposition, but this deposition was not found exclusively within the tubules but across the whole of the dentine surface. Similar results were found for the dentine samples treated with electrospun mats containing 33 wt % ACP.

Acid Challenge Studies

SEM images of dentine samples were obtained after treatment with an electrospun mat (33 wt % ACP) for 1 hour, followed by acid etching for 15 minutes. These images show that the small amount of calcium phosphate material deposited was not substantive under acid challenge; the majority of the dentinal tubules were empty and there was minimal particulate matter observed on the surface.

SEM images of dentine samples were obtained after treatment with an electrospun mat (60 wt % ACP) for 1 hour, followed by acid etching for 15. The images show material deposited within the dentinal tubules, however, there are also small areas within the tubules which are free from material. By comparing SEM images of the dentine surface before the citric acid etching stage, it can be observed that a small amount of deposited material within the tubules had been removed following the acid etch. These images indicate that the calcium phosphate material deposited within the tubules during the treatment stage was relatively substantive under acid challenge. The dentine exhibited discreet depositions of material within the tubules even after acid challenge for 15 minutes.

Mechanical Challenge Studies

An SEM image of a dentine sample was obtained after treatment with an electrospun (10 wt % ACP) mat for 1 hour, followed by mechanical challenge for 2 minutes. This image indicates that the small amount of calcium phosphate material deposited on the surface was not substantive under mechanical challenge; the dentinal tubules were empty and there was minimal particulate matter observed on the surface.

SEM images of dentine samples were obtained after treatment with an electrospun (60 wt % ACP) mat for 1 hour, followed by mechanical challenge for 2 minutes. A small amount of material was observed on the surface of the dentine however, the tubules themselves were observed to be void of deposited material. This suggests that the calcium phosphate deposited within the tubules during treatment is not substantive under a high level of mechanical challenge.

SEM images of dentine samples were obtained after treatment with an electrospun (50 wt % Sr-ACP) mat for 1 hour, followed by mechanical challenge for 2 minutes. These images show areas of dentine where material remains deposited within the tubules and areas where the tubules are empty. This suggests that there is a small level of resistance to mechanical challenge.

Hydraulic Conductance Studies

The rate of fluid flow within the dentinal tubules was investigated using Hydraulic Conductance (HC) to establish whether the calcium phosphate deposits within the tubules lead to a decrease in fluid flow.

TABLE 2
Table to show the hydraulic conductance of dentine tubules
before and after treatment with 0 wt % ACP mat

| | Flow rate/cm.min-1 | | | |
|---|---|---|---|---|
| | a. | b. | c. | Average |
| Before | 3.5 | 2.4 | 1.5 | 2.5 |
| electrospun mat | | | | |
| treatment | | | | |
| After electrospun | 2.1 | 3.0 | 3.9 | 3.0 |
| mat treatment | | | | |

TABLE 3
Table to show the hydraulic conductance of dentine tubules
before and after treatment with 60 wt % ACP mat

| | Flow rate/cm.min-1 | | | |
|---|---|---|---|---|
| | a. | b. | c. | Average |
| Before | 15.4 | 15.3 | 12.2 | 14.3 |
| electrospun mat | | | | |
| treatment | | | | |
| After electrospun | 0.3 | 0.1 | 0.1 | 0.2 |
| mat treatment | | | | |

The flow rate of fluid through dentine samples before and after treatment with an electrospun mat containing 0 wt % ACP was measured and the results are shown in Table 2. As expected, the results indicate that there was a minimal effect on the average flow rate of fluid through the tubules with respective values for before and after treatment of 2.5 and 3.0 cm.min-1. This result can be explained through the absence of any calcium phosphate material within the electrospun mats to occlude tubules and impede fluid flow through them.

Table 3 shows the results of the HC measurements taken to measure the flow rate of fluid through dentine samples before and after treatment with an electrospun mat containing 60 wt % ACP. Significantly, these results show that there was a large decrease in average fluid flow rate through the tubules from 14.3 to 0.2 cm.min-1 after treatment. This indicates that the calcium phosphate present within the electrospun mats is deposited within the dentinal tubules during treatment, therefore impeding fluid flow through them.

Dissolution Rate of Electrospun Mats

The rate of dissolution of electrospun mats was investigated using PVP polymer of varying molecular weights. The first experiments investigated the rate of dissolution of PVP powders and the results are shown in Graph 2 and indicated by the top line on the graph. It can be observed that the dissolution time increase with molecular weight.

Further to this preliminary experiment, electrospun mats of PVP with varying molecular weight were fabricated (as detailed in the experimental section of this report) and the dissolution rate of these materials were investigated. The results are shown in Graph 2 and are indicated by the bottom line on the graph. As would be expected, the dissolution rates of the electrospun PVP mats in water followed the same trend as the PVP powders, with higher molecular weights of PVP leading to higher dissolution rates.

These results indicate that the rate of dissolution of the electrospun mats can be altered using the molecular weight of the polymer to achieve the desired rate. This suggests that the rate of delivery of ACP to the tooth could therefore be tunable.

### CONCLUSIONS

The research discussed in this example confirms that electrospun mats of ACP are able to effectively occlude dentinal tubules as shown through SEM images and Hydraulic conductance (HC) data. The SEM images show discreet occlusion of the dentinal tubules when treated with electrospun mats of 60 wt % ACP for one hour and the HC data confirmed that treatment with these calcium phosphate mats reduced the fluid flow rate through the dentinal tubules significantly.

The calcium phosphate material within the tubules was observed to be relatively substantive under acidic challenge, however, an optimal amount of ACP was needed within the mats to maximise calcium phosphate deposition and tubule occlusion; electrospun mats with ACP contents of 33 wt % and below were observed to be less effective at providing substantial tubular occlusion.

The rate of dissolution of the electrospun mats in water solutions was observed to increase with increasing molecular weights of polymer used. This result suggests that the dissolution of the mats and therefore the rate of delivery of ACP to the tooth can be tuned.

1. An electrospun polymer fibre comprising an amorphous calcium compound.

2. A fibre according to claim 1 wherein the amorphous calcium compound is amorphous calcium phosphate or amorphous strontium calcium phosphate or a mixture thereof.

3. A fibre according to claim 1 wherein the polymer is a polyvinyl pyrrolidone (PVP) or a derivative thereof, a polysaccharide, a cellulose polymer, an anionic polymer, a biopolymer, a bioerodible polymer, a polyethylene oxide, a polyvinyl alcohol or an acrylamide copolymer or a mixture thereof.

4. A fibre according to claim 3 wherein the polymer is PVP or a derivative thereof.

5. A fibre according to claim 1 further comprising an oral care active agent.

6. An oral care composition comprising a fibre according to claim 1.

7. A composition according to claim 6 further comprising an oral care active agent.

8. A composition according to claim 6 which is an anhydrous composition.

9. (canceled)

10. A method of remineralising dental hard tissues and/or blocking dentinal tubules in a patient in need thereof which comprises administering an effective amount of an oral composition according to claim 1.
